(19) United States
(12) Patent Application Publication
(10) Pub. No.: US 2015/0320738 A1
(43) Pub. Date: Nov. 12, 2015

(54) BERBERINE-CONTAINING PHARMACEUTICAL COMPOSITION FOR INHIBITING CANCER STEM CELL GROWTH OR CARCINOMA METASTASIS AND APPLICATION THEREOF

(71) Applicants: Chen-Yu Lee, Taipei City (TW); National Taiwan Normal University, Taipei City (TW)

(72) Inventors: Hsiu-Mei HSIEH, Taipei City (TW); Chen-Yu LEE, Taipei City (TW); Chih-Chien SHEN, Keelung City (TW); Tien-Chun WANG, New Taipei City (TW)

(21) Appl. No.: 14/790,154

(22) Filed: Jul. 2, 2015

Related U.S. Application Data
(62) Division of application No. 13/137,711, filed on Sep. 7, 2011.

(30) Foreign Application Priority Data
Jun. 14, 2011 (TW) 100120648

(51) Int. Cl. A61K 31/4745; A61K 36/718

(52) U.S. Cl. CPC A61K 31/4745; A61K 36/718

(57) ABSTRACT

A pharmaceutical composition for inhibiting cancer stem cells growth or carcinoma metastasis and an application thereof are disclosed. The pharmaceutical composition includes: a berberine compound; and a pharmaceutically acceptable carrier. The application is the use of the berberine compound in the manufacture of a medicament for the inhibition of cancer stem cell growth or carcinoma metastasis.

### CROSS REFERENCE TO RELATED APPLICATION

This application is a divisional application of pending U.S. patent application Ser. No. 13/137,711, filed on Sep. 7, 2011, which claims priority to Taiwan application No. 100120648, filed on Jun. 14, 2011 (of which the entire disclosures of the pending, prior application is hereby incorporated by reference).

### BACKGROUND OF THE INVENTION

1. Field of the Invention

The present invention relates to a pharmaceutical composition and application for inhibiting cancer stem cell growth or carcinoma metastasis and, more particularly, to a berberine compound-containing pharmaceutical composition for inhibiting cancer stem cell growth or carcinoma metastasis and use of a berberine compound in the manufacturing of a medicament for the inhibition of cancer stem cell growth or carcinoma metastasis.

2. Description of Related Art

Cancer becomes the top factor of the ten major factors of death in past 27 years. The main factor of cancer growing is that cells begin to self-proliferate and become abnormal to form more and more abnormal cells, i.e. cancer.

In common tumor cells, researchers found that some cancer cells have characteristics of stem cells. Although such cancer cells are few, they act as stem cells and are able to proliferate and differentiate. Hence, they are named “cancer stem cells”. Because cancer stem cells have extremely high resistance to drugs, it is difficult for chemotherapeutic agents of modern (Western) medicine to exterminate them. Accordingly, it is often heard that cancer recurrence happens in many patients treated with chemotherapy. In addition, standard therapies currently known in biomedical science are still unable to kill such cancer stem cells.

Furthermore, surgical operations, radiotherapies, chemotherapies, hormone therapies, biological therapies, and so on in modern medical science may incur strongly unfavorable side effects in patients. Therefore, it is a significant breakthrough if cancer can be treated by a therapy that is relatively gentle and able to inhibit development of cancer stem cells.

Currently, people believe that the use of Chinese herbal medicine to treat patients is a gentle therapy and highly acceptable in commerce, and it has become a complementary and alternative medicine. If one of various Chinese herbal medicines can be screened and evidenced to inhibit carcinoma metastasis and block proliferation of cancer stem cells, it will be considerably helpful to the treatment of cancer.

### SUMMARY OF THE INVENTION

The object of the present invention is to provide a pharmaceutical composition of inhibiting cancer stem cell growth or carcinoma metastasis. The pharmaceutical composition can be used to significantly decrease viability of cancer cells such as non-small-cell lung carcinoma (NSCLC) cells, but will not undesirably influence normal cells in an effective amount. Also the pharmaceutical composition can effectively inhibit carcinoma metastasis.

Another object of the present invention is to provide use of a berberine compound in the manufacturing of a medicament for the inhibition of cancer stem cell growth or carcinoma metastasis. The medicament can be used to decrease proliferation and metastasis of cancer cells and the ratio of cancer stem-like cells and includes health food products and clinically therapeutic drugs used for the prophylaxis and therapy of cancer.

In order to achieve the objects depicted above, one aspect of the present invention provides a pharmaceutical composition of inhibiting cancer stem cell growth or carcinoma metastasis, which includes a berberine compound and a pharmaceutically acceptable carrier.

Another aspect of the present invention provides use of a berberine compound in the manufacturing of a medicament for the inhibition of cancer stem cell growth or carcinoma metastasis.

In the above-mentioned pharmaceutical composition and use of the present invention, the berberine compound can be purchased commercially. Berberine chloride and its hydrate, berberine sulfate, berberine hemisulfate, berberine bisulfate, and so on are exemplified as the berberine compound. Otherwise, the berberine compound can be obtained from extraction of an herbal material of Coptis chinensis.

When the herbal material of C. chinensis is extracted, the berberine compound is present in the extract of the herbal material of C. chinensis. For example, a herbal material of C. chinensis is added with water in an amount of 50-200 times the weight of the herbal material to form a mixture, and then the mixture is extracted under heating for 30 minutes to 2 hours or until the volume of the mixture is changed into one-fourth to half the original volume of the mixture, so as to give a water extract of C. chinensis. Thus, the water extract of C. chinensis contains the berberine compound, and can be processed by a drying method such as spray-drying, lyophilization, and granulation of scientifically concentrated traditional Chinese medicines to form a dry extract.

Accordingly, a pharmaceutical composition containing a berberine compound and used to inhibit cancer stem cell growth or carcinoma metastasis, a method of inhibiting cancer stem cell growth or carcinoma metastasis, and use of a berberine compound in the manufacture of a medicament for the inhibition of cancer stem cell growth or carcinoma metastasis are construed in the scope of the present invention. The cancer stem cells or carcinoma cells can be non-small-cell lung carcinoma (NSCLC) cells.

In conclusion, in the present invention, the aforesaid pharmaceutical composition and the use of the berberine compound in the manufacture of a medicament for the inhibition of cancer stem cell growth and carcinoma metastasis can pass through a bottleneck of a conventional treatment that is not able to efficiently inhibit cancer stem cells. Therefore, health food products and clinically therapeutic drugs can be developed for the prophylaxis and therapy of cancer.

Other objects, advantages, and novel features of the invention will become more apparent from the following detailed description when taken in conjunction with the accompanying drawings.

### BRIEF DESCRIPTION OF THE DRAWINGS

FIG. 1 shows cell viability of A549, NCI-H460, NCI-H520, and MRC5 cells after they are treated with the extract of C. chinensis;

FIG. 2A shows a result of MTT test in which A549 cells are treated with a berberine solution for 48 hours;

FIG. 2B shows a result of MTT test in which NCI-H460 cells are treated with a berberine solution for 48 hours;

FIG. 2C shows a result of MTT test in which NCI-H520 cells are treated with a berberine solution for 48 hours;

FIG. 3 shows a result of trypan blue test in which A549 cells are treated with the extract of C. chinensis;

FIG. 4 shows the cell cycle of A549 cells that are treated with the extract of C. chinensis;

FIG. 5A shows the expression of CDK4 after the treatment of the extract of C. chinensis;

FIG. 5B shows the expression of CDK6 after the treatment of the extract of C. chinensis;

FIG. 5C shows the expression of Cyclin D3 after the treatment of the extract of C. chinensis;

FIG. 5D shows the expression of Cyclin B3 after the treatment of the extract of C. chinensis;

FIG. 5E shows the expression of Vimentin after the treatment of the extract of C. chinensis;

FIG. 5F shows the expression of ALDH1A1 after the treatment of the extract of C. chinensis;

FIG. 5G shows the expression of β-catenin after the treatment of the extract of C. chinensis;

FIG. 5H shows the expression of ABCG2 after the treatment of the extract of C. chinensis;

FIG. 6 shows the cell number per captured view by microscope from 15 views in total in a transwell assay, where the cells are treated with the extract of C. chinensis;

FIG. 7A shows characterization of side population (SP) in A549 cells treated with the extract of C. chinensis; and

FIG. 7B shows characterization of side population (SP) in A549 cells treated with the berberine solution.

### DETAILED DESCRIPTION OF THE PREFERRED EMBODIMENT

In the present invention, the extract obtained from an herbal material of C. chinensis is examined by a series of bioassays and found that it is able to inhibit cancer stem cells and carcinoma metastasis. Also, the extract of C. chinensis is preliminarily analyzed in regard to its components and found that the major component is berberine. Thus, the inventors infer that berberine should achieve the same effects delineated above. Therefore, the inventors purchase berberine commercially and prepare a berberine solution for bioassays. Accordingly, it is confirmed that the extract of C. chinensis and berberine are both able to inhibit cancer stem cells and carcinoma metastasis.

As used herein, the term “inhibiting” refers to administering a pharmaceutical composition containing a berberine compound to a subject that has cancer, or has a symptom of or a predisposition toward it, with the purpose to cure, heal, alleviate, relieve, alter, remedy, ameliorate, improve, or affect the cancer, the symptoms of or the predisposition toward it. The term “an effective amount” refers to the amount of the active agent that is required to confer the intended therapeutic effect in the subject. The effective amount may vary, as recognized by those skilled in the art, depending on route of administration, excipient usage, and the possibility of co-usage with other agents.

Cancer that can be treated by the method of the present invention includes both solid and hematological tumors of various organs. Examples of solid tumors include pancreatic cancer; bladder cancer; colorectal cancer; breast cancer, including metastatic breast cancer; renal cancer, including, e.g., metastatic renal cell carcinoma; hepatocellular cancer; lung cancer, including, e.g., non-small cell lung cancer (NSCLC), bronchioloalveolar carcinoma (BAC), and adenocarcinoma of the lung; prostate cancer, including androgen-dependent and androgen-independent prostate cancer; ovarian cancer, including, e.g., progressive epithelial or primary peritoneal cancer; neuroendocrine cancer, including metastatic neuroendocrine tumors; brain tumors, including, e.g., glioma, anaplastic oligodendroglioma, adult glioblastoma multiforme, and adult anaplastic astrocytoma; cervical cancer; gastric cancer; esophageal cancer; head and neck cancer, including, e.g., squamous cell carcinoma of the head and neck; melanoma; bone cancer; and soft tissue sarcoma. Examples of hematological malignancy include acute myeloid leukemia (AML); chronic myelogenous leukemia (CML), including accelerated CML and CML blast phase (CML-BP); myelodysplastic syndromes (MDS), including refractory anemia (RA), refractory anemia with ringed siderblasts (RARS), (refractory anemia with excess blasts (RAEB), and RAEB in transformation (RAEB-T); non-Hodgkin's lymphoma (NHL), including follicular lymphoma and mantle cell lymphoma; B-cell lymphoma; T-cell lymphoma; multiple myeloma (MM); acute lymphoblastic leukemia (ALL); chronic lymphocytic leukemia (CLL); Hodgkin's disease (HD); Waldenstrom's macroglobulinemia; and myeloproliferative syndromes.

The pharmaceutical composition of the present invention can further include a therapeutic agent such as a cytotoxic agent, or be applied in combination with another therapy such as radiotherapy and immunotherapy. For example, the cytotoxic agent can be antimetabolites, including, e.g., capecitibine, gemcitabine, 5-fluorouracil or 5-fluorouracil/leucovorin, fludarabine, cytarabine, mercaptopurine, thioguanine, pentostatin, and methotrexate; topoisomerase inhibitors, including, e.g., etoposide, teniposide, camptothecin, topotecan, irinotecan, doxorubicin, and daunorubicin; vinca alkaloids, including, e.g., vincristine and vinblastin; taxanes, including, e.g., paclitaxel and docetaxel; platinum agents, including, e.g., cisplatin, carboplatin, and oxaliplatin; antibiotics, including, e.g., actinomycin D, bleomycin, mitomycin C, adriamycin, daunorubicin, idarubicin, doxorubicin and pegylated liposomal doxorubicin; alkylating agents such as melphalan, chlorambucil, busulfan, thiotepa, ifosfamide, carmustine, lomustine, semustine, streptozocin, decarbazine, and cyclophosphamide; thalidomide and related analogs, including, e.g., CC-5013 and CC-4047; protein kinase inhibitors, including, e.g., imatinib mesylate, gefitinib, dasatinib, erlotinib, lapatinib, sunitinib, nilotinib, and sorafenib; antibodies, including, e.g., trastuzumab, rituximab, cetuximab, and bevacizumab; mitoxantrone; dexamethasone; prednisone; and temozolomide.

In order to practice the method described in the present invention, the above-mentioned pharmaceutical composition can be administered orally, parenterally, by inhalation spray, topically, rectally, nasally, buccally, vaginally or via an implanted reservoir. The term “parenterally” as used herein includes subcutaneous, intracutaneous, intravenous, intramuscular, intraarticular, intraarterial, intrasynovial, intrasternal, intrathecal, intralesional, and intracranial injection or infusion techniques. A sterile injectable composition, e.g., a sterile injectable aqueous or oleaginous suspension, can be formulated according to techniques known in the art using suitable dispersing or wetting agents (such as Tween 80) and suspending agents. The sterile injectable preparation can also be a sterile injectable solution or suspension in a non-toxic parenterally acceptable diluent or solvent, for example, as a solution in 1,3-butanediol.

Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or diglycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purposes of formulation.

A composition for oral administration can be any orally acceptable dosage form including, but not limited to, capsules, tablets, emulsions and aqueous suspensions, dispersions and solutions. In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions or emulsions are administered orally, the active ingredient can be suspended or dissolved in an oily phase combined with emulsifying or suspending agents. If desired, certain sweetening, flavoring, or coloring agents can be added. A nasal aerosol or inhalation composition can be prepared according to techniques well known in the art of pharmaceutical formulation. For example, the composition can be processed into a salt solution added with benzyl alcohol or other suitable preservatives, absorbefacients to enhance bioavailability, carbon fluorides, and/or dissolving or dispersing agents known in the art of the present invention. The pharmaceutical composition containing a berberine compound can also be administered in the form of suppositories for rectal administration.

The carrier in the pharmaceutical composition must be “acceptable” in the sense of being compatible with the active ingredient of the formulation (and preferably, capable of stabilizing it) and not deleterious to the subject to be treated. One or more solubilizing agents (e.g., cyclodextrins) which form more soluble complexes with the active berberine compound can be utilized as pharmaceutical carriers for delivery of the active compounds. Examples of other carriers include colloidal silicon dioxide, magnesium stearate, sodium lauryl sulfate, and D&C Yellow #10.

Because of the specific embodiments illustrating the practice of the present invention, one skilled in the art can easily understand other advantages and efficiency of the present invention through the content disclosed therein. The present invention can also be practiced or applied by other variant embodiments. Many other possible modifications and variations of any detail in the present specification based on different outlooks and applications can be made without departing from the spirit of the invention.

Preparation of an Extract of C. chinensis

11.25 g of C. chinensis (CC) were mixed with water in 1.2 L and boiled for 1 hour to give an extract (about 450 ml). The extract was divided into aliquots and stored at 4° C. In addition to the aforesaid storage of the water extract at 4° C., the extract could be lyophilized before being stored.

Before the following experiments, the extract stored at 4° C. could be filtered with 0.22 μm membranes.

### Preparation of Berberine Solutions

Dimethyl sulfoxide (DMSO) was used as a solvent to dissolve berberine chloride (B3251, purchased from Sigma) and thus berberine solutions were prepared in different concentrations.

### Cell Culture

NSCLC cell lines A549, NCI-H460, NCI-H520 and normal lung cell line MRC5 were obtained from Bioresource Collection and Research Center (BCRC, Taiwan). A549 cell line was cultured with F12K medium (21127, GIBCO, Carlsbad, U.S.A.). Both NCI-H460 and NCI-H520 cell lines were cultured with RPMI1640 medium (23400, GIBCO, Carlsbad, U.S.A.). MRC5 cell line was culture with MEM medium (41500, GIBCO, Carlsbad, U.S.A.). All media mentioned above contained 10% FBS (10437, GIBCO, Carlsbad, U.S.A.). All cultures were maintained in an incubator (Thermo forma 370, Waltham, U.S.A.) with 5% CO₂ at 37° C.

### High Performance Liquid Chromatography (HPLC)

The HPLC system (Hitachi) with reverse-phase Zorbax ODS II (150 mm×4.6 mm, 5 μm) column was used for CC analysis., the sample injection volume was 10 μl, and the analysis was performed at a column temperature of 40° C. and flow rate of 1.0 ml/min. The UV absorbance of the eluate was measured at 346 nm.

Potassium hydrogen phosphate (1.36 g) was dissolved in 1000 ml water and then adjusted to pH 2.5 with orthophosphoric acid to form buffer (A). The ratio of buffer (A) to acetonitrile (used as buffer B) was adjusted according to the following gradient: 0.01 min, A:B=80:20→20 min, A:B=50:50→26 min, A:B=80:20.

The berberine solution and extract of C. chinensis were analyzed by HPLC. The result showed that the peak of berberine was found at the retention time of about 9.5 minutes. Also, a large peak of the extract of C. chinensis was found at the retention time of about 9.5 minutes. This result means the extract of C. chinensis contains a great amount of berberine.

### Statistic Analysis

All data from the following experiments were presented as mean±SE. Student's test and ANOVA were used with SPSS software to determine the significance of differences depending on the number of groups. In the test, p-value<0.05 was considered as a significant difference.

### Test Example 1

### Cell Proliferation Assay

Cells were seeded at 1×10⁴ cells per well in a 24-well plate for 16 hours. After that, a sample such as the berberine solutions with different concentrations and the extract of C. chinensis was added into 24-well plate directly, and cultured at 37° C. for 24, 48, and 72 hours, respectively.

### I. MTT Assay

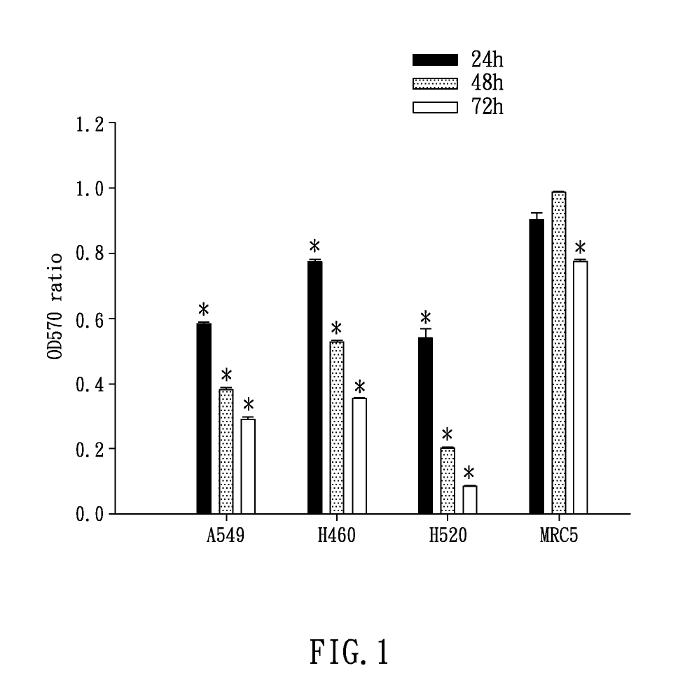

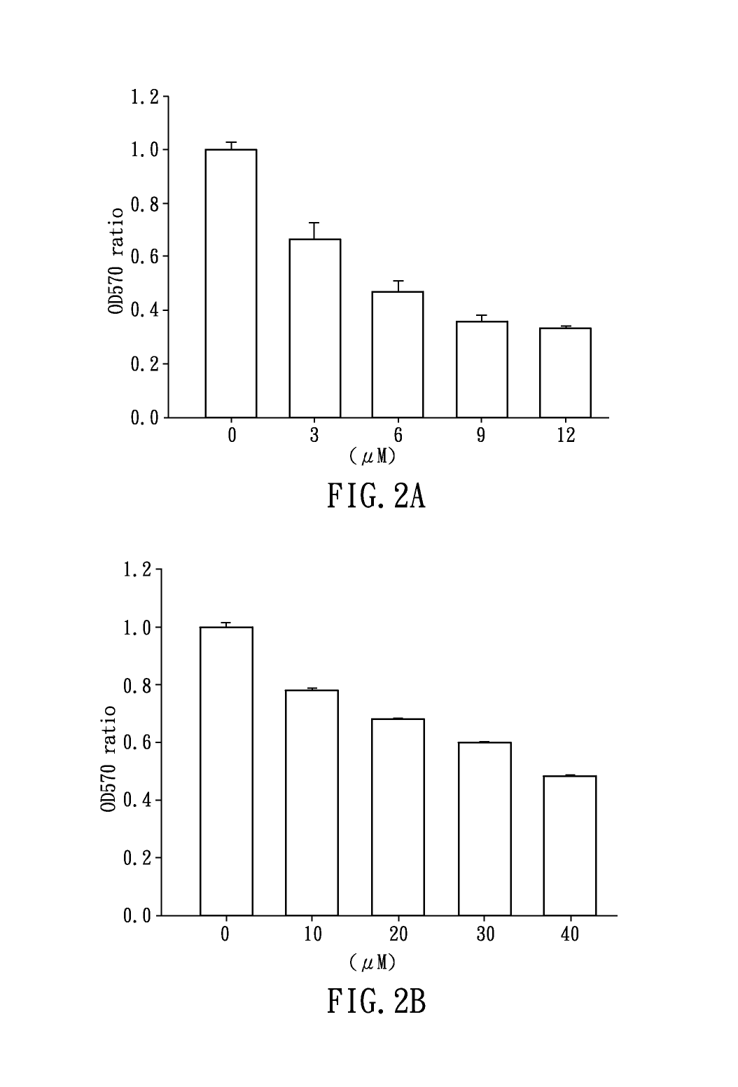

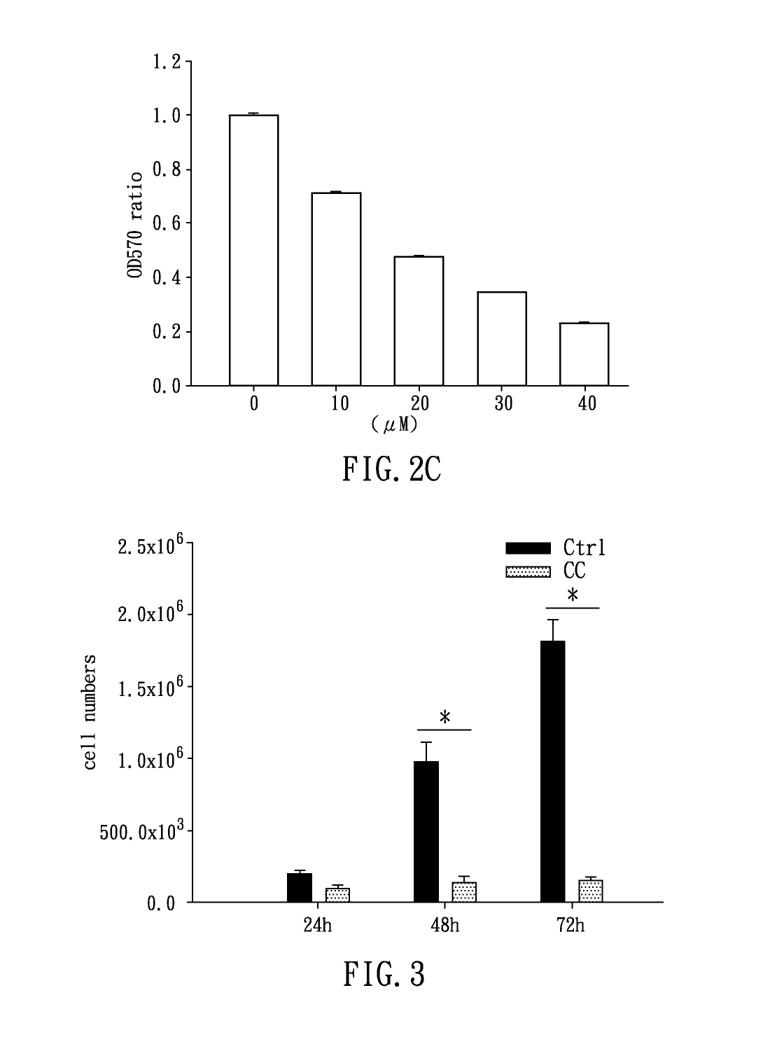

Thiazolyl blue tetrazolium bromide (MTT, m5655, Sigma, St. Louis, U.S.A.) solution was prepared in a concentration of 5.5 mg/ml with phosphate buffered saline (PBS) filtered by 0.22 μm membrane. The MTT solution (50 μl) was added to the wells of 24-well plates with A549, NCI-H460, NCI-H520, and MRC5 cell lines cultured after 24, 48, and 72 hours. The plates were incubated at 37° C. for 2 hours and then moved out. The media of the wells were removed. Subsequently, DMSO (500 μl) was added into each well to dissolve the products, formazan dyes, from the reaction of MTT and dehydrogenase in mitochondria. After the formazan dyes were fully dissolved in the plate by pipetting, the resultant solution (200 μl) was transferred into a 96-well ELISA plate. Then, microplate autoreader EL311 (Bio-Tek Instruments, Winooski, U.S.A.) was used to detect the absorption (O.D. 570 nm) of the solution. The results are shown in FIGS. 1 and 2A to 2C.

With reference to FIG. 1, it shows the result of A549, NCI-H460, NCI-H520, and MRC5 cell lines treated with the extract of C. chinensis (0.6% v/v), and * represents p<0.05. In FIG. 1, it can be seen that compared with the viability of normal cells, that of cancer cells is considerably decreased after they are treated with the extract of C. chinensis for 48 hours. When the time of the treatment is extended to 72 hours, the viability of the cancer cells is significantly decreased. This result indicates that the extract of C. chinensis of the present invention is able to reduce cancer cells and decrease their viability without influencing normal cells.

With reference to FIG. 2A, it shows the result of A549 cell line treated with the berberine solution (3, 6, 9, and 12 μM) for 48 hours. In FIG. 2A, it can be understood that the inhibition of A549 cell line is increased as the concentration of the berberine solution increases, and its half maximal inhibitory concentration (IC₅₀) is approximately 7 μM.

With reference to FIG. 2B, it shows the result of NCI-H460 cell line treated with the berberine solution (10, 20, 30, and 40 μM) for 48 hours. In FIG. 2B, it can be seen that the inhibition of NCI-H460 cell line is increased as the concentration of the berberine solution increases, and its half maximal inhibitory concentration (IC₅₀) is approximately 40 μM.

With reference to FIG. 2C, it shows the result of NCI-H520 cell line treated with the berberine solution (10, 20, 30, and 40 μM) for 48 hours. In FIG. 2C, it can be seen that the inhibition of NCI-H520 cell line is increased as the concentration of the berberine solution increases, and its half maximal inhibitory concentration (IC₅₀) is approximately 20 μM.

According to FIGS. 2A to 2C, it can be known that the berberine compound is similar to the extract of C. chinensis of the present invention, and both of them are able to reduce cancer cells and decrease their viability.

### II. Trypan Blue Assay

Trypan blue (T10282, Invitrogen, Carlsbad, U.S.A.) was used to stain the A549 cell line treated with the extract of C. chinensis (0.6% v/v). Then, the staining solution of the cells was transferred into Countess® Cell Counting Chamber Slide (C10228, Invitrogen, Carlsbad, U.S.A.), and Countess® Automated Cell Counter (C10227, Invitrogen, Carlsbad, U.S.A.) was further used to detect the number of cells. The result is shown in FIG. 3.

With reference FIG. 3, it shows the result of A549 cell line treated with the extract of C. chinensis (0.6% v/v), and * represents p<0.05. In FIG. 3, it can be seen that compared with the viability of the control group (ctrl, i.e. an untreated group), that of cancer cells is considerably decreased after they are treated with the extract of C. chinensis for 48 hours. When the time of the treatment is extended to 72 hours, the viability of the cancer cells is significantly decreased. This result indicates that the extract of C. chinensis of the present invention is able to reduce cancer cells and decrease their viability without influencing normal cells.

### Test Example 2

### Cell Cycle Analysis

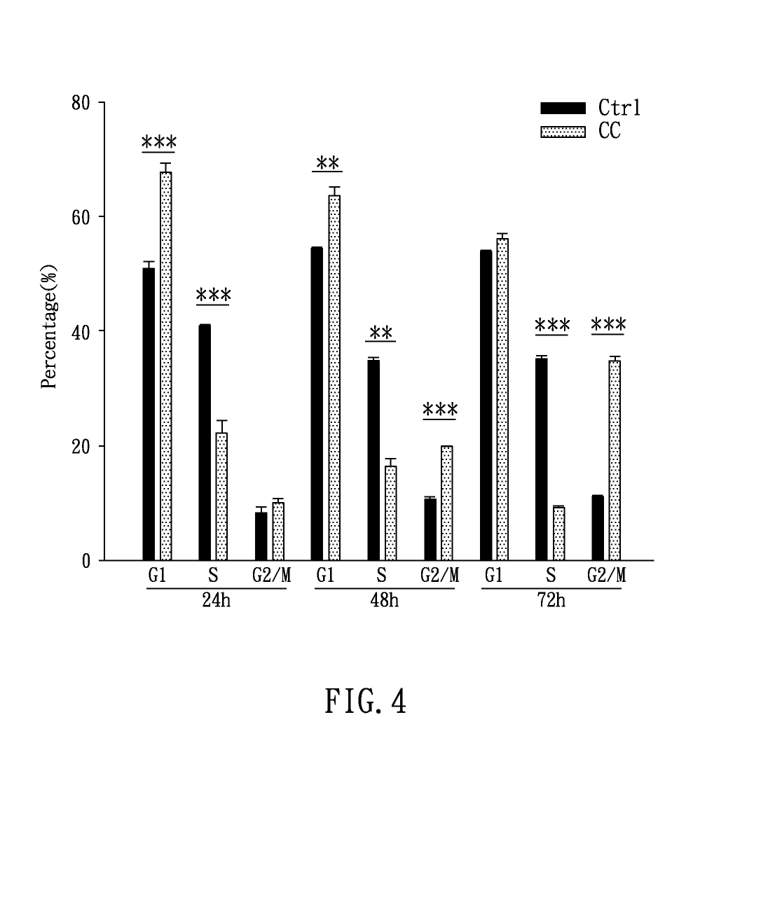

A549 cells were seeded in the number of about 3×10⁵ in 10 cm dish to give adequate number of cells for analysis. When the cells were analyzed, they could maintain logarithmical growth. After seeding for 16 hours, the cells were treated with the extract of C. chinensis for 24, 48, and 72 hours, and then harvested by trypsinization. The harvested cells were fixed with cold EtOH (70%) at −20° C. overnight. Subsequently, the cells were washed with PBS to remove the excess EtOH. PI staining buffer (PBS: RNase (10 μg/ml): PI (1 μg/ml)=97: 1: 2) was added to the cells (1 ml for 1×10⁶ cells) to ensure a thorough staining of DNA. The staining was conducted at 37° C. in the dark for 30 min under frequent shaking. Nylon mesh (35 μm) was used to filter the cells to prevent the cell clumps. The resultant samples were then transferred into round bottom tubes and analyzed with FACSCalibur as soon as possible. Single cell gate was created to exclude aggregated cells. 50 thousand cells per sample were collected to convey the cell cycle distribution. Modfit software (Verity Software House, Topsham, U.S.A.) was used to later calculate the percentages of different cell cycle stages. The result is shown in FIG. 4.

With reference FIG. 4, it shows the result of the cell cycle assay on A549 cell line treated with the extract of C. chinensis, and * and *** respectively denote p<0.05 and p<0.001. In FIG. 4, it can be seen that the cell cycle stage of A549 cell line treated with the extract of C. chinensis for 24 hours is stopped clearly at G1 phase. When the time of the treatment is extended to 48 hours, the cell cycle stage of A549 cell line is blocked obviously at G2 phase. This result indicates the extract of C. chinensis of the present invention is able to inhibit continuous cell division of cancer cells.

### Test Example 3

### Western Blot Assay

A549 cells were seeded in the number of about 3×10⁵ in 10 cm dish to give enough number of cells for analysis. When the cells were analyzed, they could keep at logarithmical growth. The cells were treated with the extract of C. chinensis or the berberine solution with the dosage of IC₅₀ for 48 hours. The treated cells were collected, lysed with RIPA buffer (10 mM Tris (pH 7.4), 150 mM NaCl, 5 mM EDTA (pH 8.0), 0.1% SDS, 1% DOS, 1% NP40), and mixed with protease inhibitor cocktail (Pierce, Rockford, U.S.A.) and phosphatase inhibitor cocktail (Sigma, St. Louis, U.S.A.). The resultant lysate was centrifuged by 13,300 g for 30 min at 4° C., so as to spin down cell debris. The supernatant was collected after centrifugation and mixed with sample buffer (100 mM Tris-Cl (pH 6.8), 4% (w/v) SDS, 0.2% (w/v) bromophenol blue, 20% (v/v) glycerol, 200 mM β-mercaptoethanol), stored at −80° C. until use.

Protein concentration was quantified by BCA protein assay kit (23250, Thermo Scientific, Waltham, U.S.A.) according to instruction of the manufacturer. Protein (20 μg) of each sample was electrophoresed with 10% to 15% SDS-PAGE, which was prepared according to the molecular weight of each protein to be detected. SDS-PAGE was later electro-transferred onto nitrocellulose (NC) membrane at 400 mA for 1 to 2 hours.

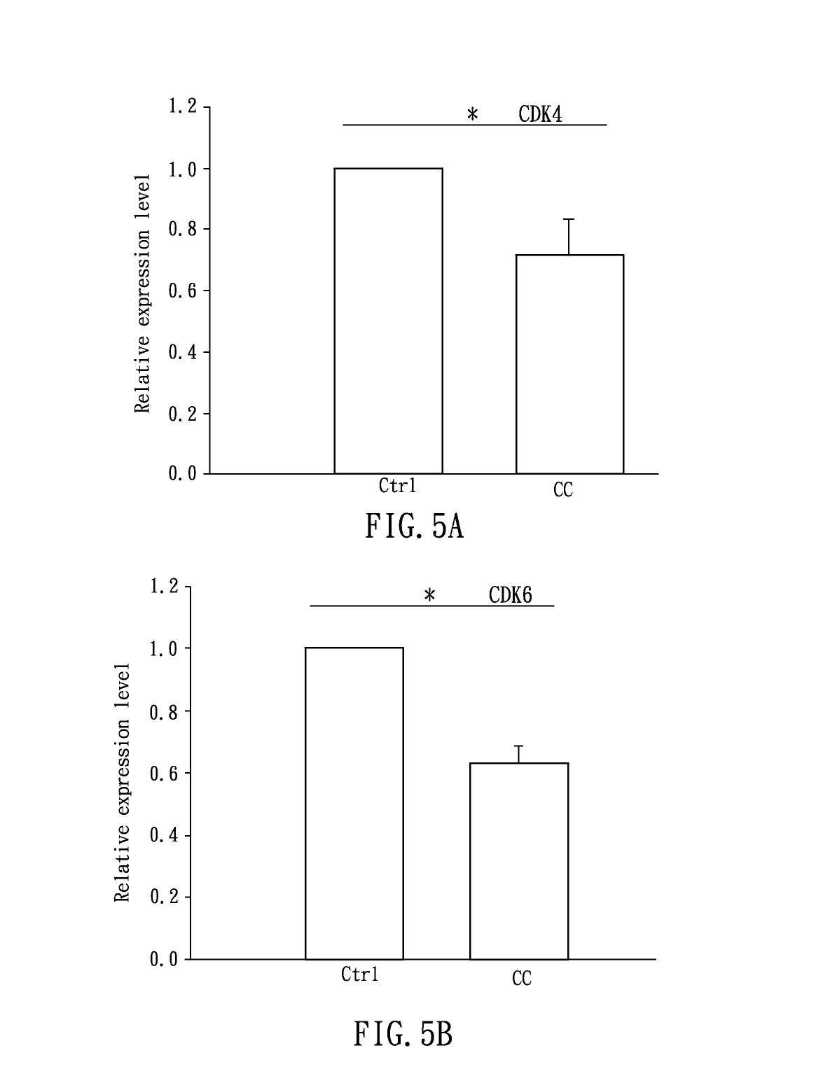

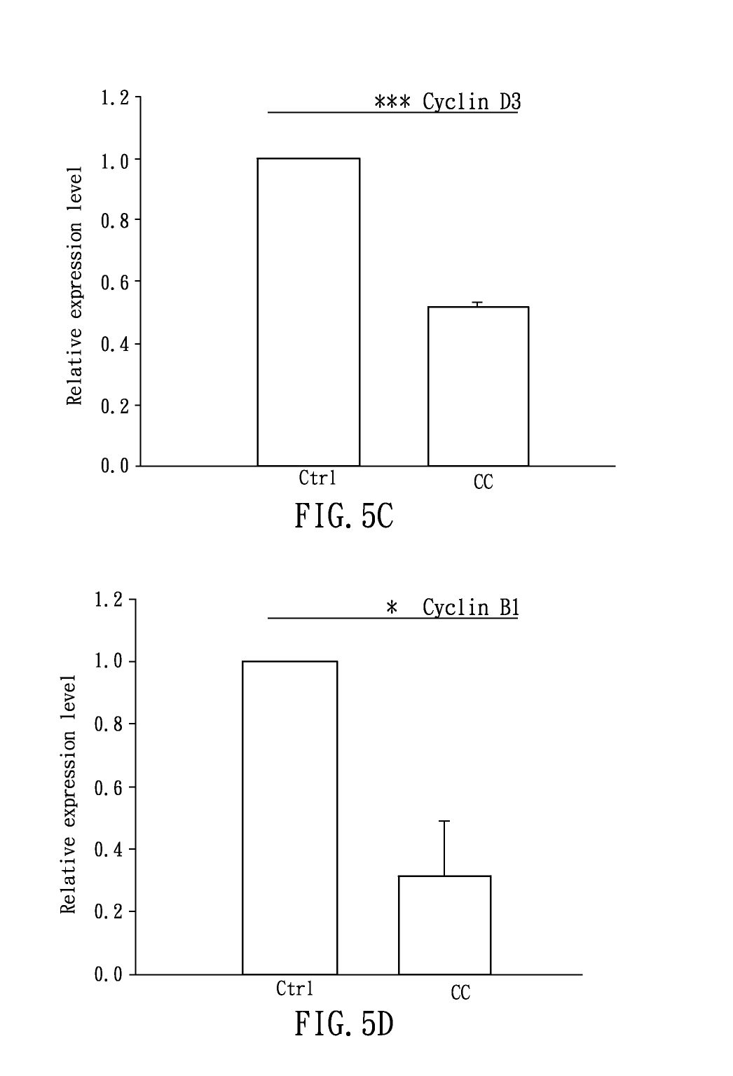

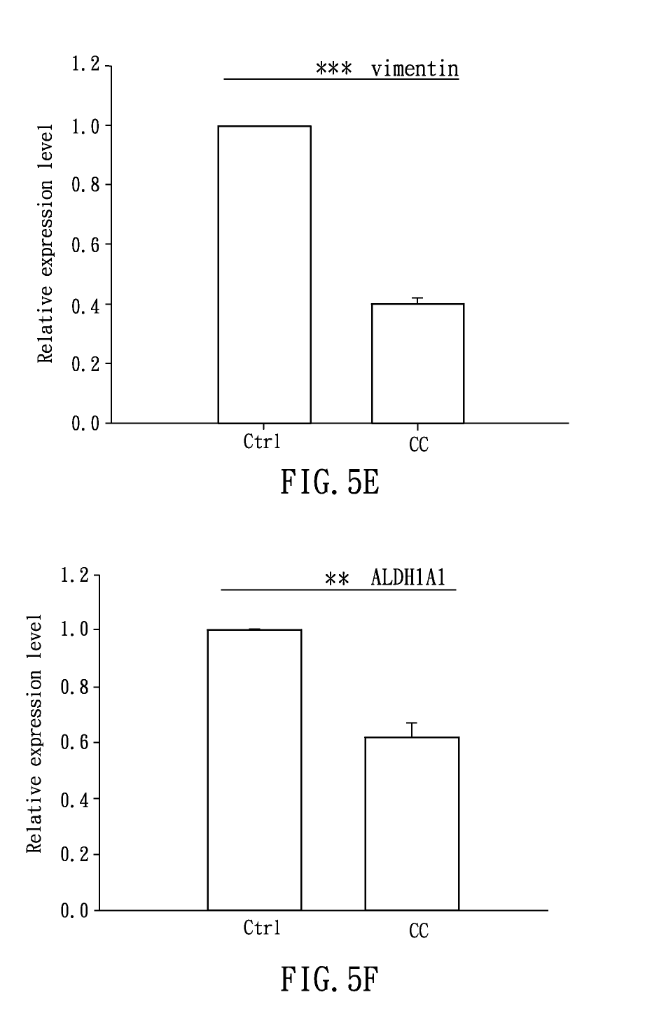

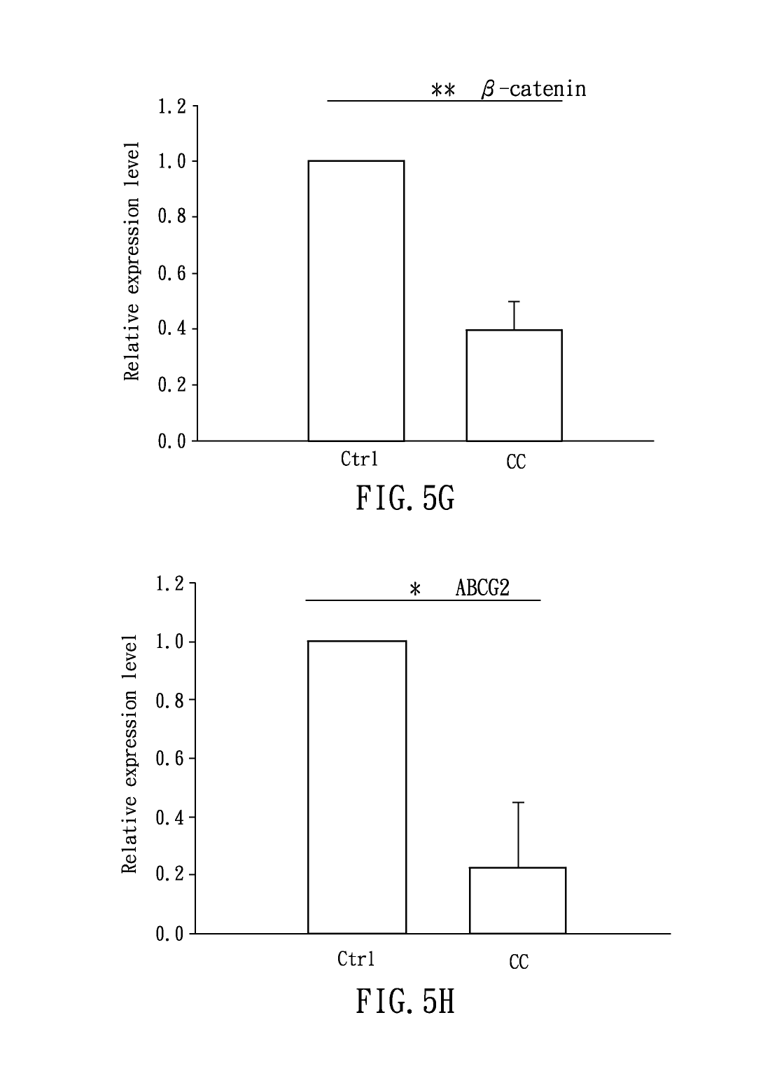

Blot membrane was later blocked by TBST (Tris Buffered Saline with 0.05% Tween-20 and 5% non-fat dry milk) for 1 hour at room temperature. The membrane was washed twice for 5 min with TBST to remove excess milk and then incubated with primary antibody in a proper dilution at 4° C. for overnight under gentle shaking. Corresponding HRP-conjugated secondary antibody was used to incubate the membrane for 1 hour after three times of washing with TBST to remove excess primary antibody. Finally, HRP substrate (WBKLS0050, Millipore, Billerica, U.S.A.) was added onto the membrane to show the pattern of protein expression before the images of the pattern were captured by Fuji LAS-3000 imaging system. The blot images were then quantified by Multi Gauge software (FUJIFILM, Tokyo, Japan). The results are shown in FIGS. 5A to 5H.

With reference to FIGS. 5A to 5H, they respectively show the expression of CDK4, CDK6, and cyclin D3 (G1/S regulators), cyclin B1 (G2/M regulator), vimentin (mesenchymal marker), and ALDH1A1, β-catenin, and ABCG2 (cancer stem cell markers) after the cancer cells are treated with the extraction of C. chinensis. Also, in these figures, *, **, and *** respectively denote p<0.05, p<0.01 and p<0.001, and the internal control is β-actin. According to FIGS. 5A to 5H, it can be known that the expression of the regulators and the markers is significantly reduced after the treatment of the extraction of C. chinensis. This result indicates the extract of C. chinensis of the present invention is able to reduce cancer cell growth and their metastasis and inhibit cancer stem cells.

### Test Example 4

### Transwell Assay

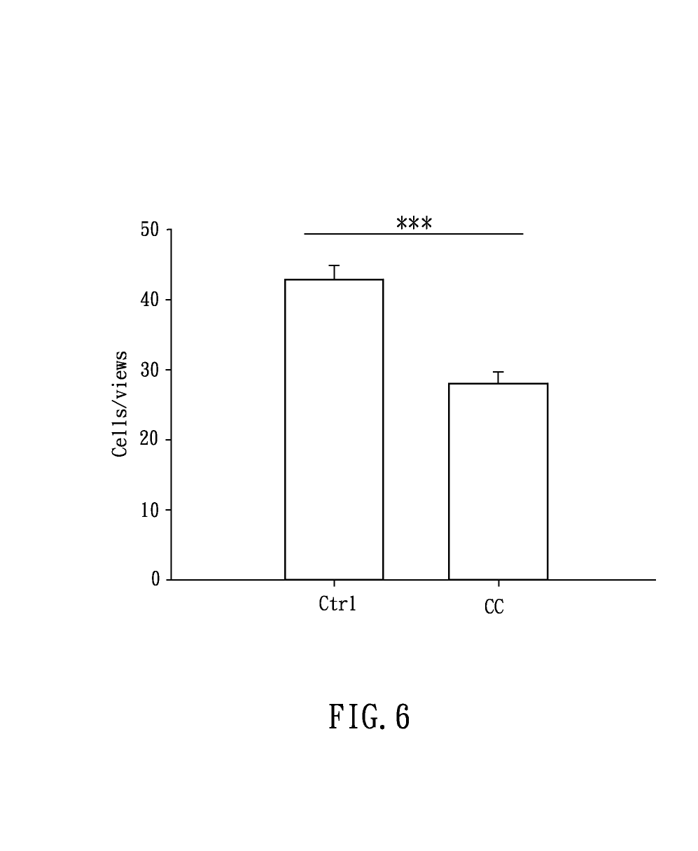

A549 cells were seeded in the number of about 2×10⁴ cells/well with serum-free medium (100 μl) in an upper part of each 6.5 mm insert with pore size 8 μm in a 24-well transwell plates (Corning, Lowell, U.S.A.), whereas the lower compartments were filled with F12K medium (500 μl) containing 10% FBS. The cells were treated with the extract of C. chinensis (0.6% v/v) for 4 hours and then fixed with paraformaldehyde (PFA) for 10 min. Subsequently, the fixed cells were stained with 4′,6-diamidino-2-phenylindol (DAPI, 1:10000 in PBST containing 0.2% Tween-20) for 10 min. The insert was washed by PBST three times each for 10 min and then observed by live cell image system (Leica, Wetzlar, Germany). The cell number on the inserts was quantified by MetaXpress software (Molecular Devices, Sunnyvale, U.S.A.). The result is shown in FIG. 6.

With reference to FIG. 6, it shows the cell number per captured view by microscope from 15 views in total after the cells are treated with the extract of C. chinensis, and *** denotes p<0.001. Referring to FIGS. 6 and 5E, it can be seen that compared with the control group, the extract of C. chinensis is able to significantly decrease carcinoma metastasis.

### Test Example 5

### Side Population Analysis

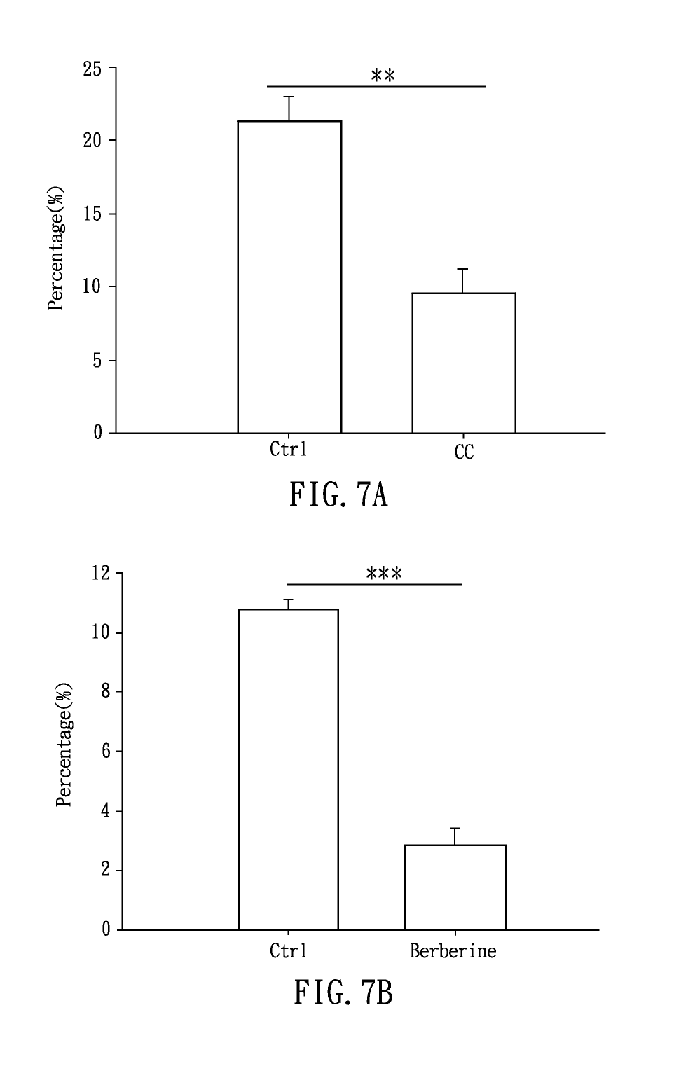

A549 cells were seeded in the number of about 3×10⁵ in 10 cm dish to give adequate number of cells for analysis. The cells were treated with the extract of C. chinensis and the berberine solution in the dose of IC₅₀ for 48 hours. Then, the treated cells were harvested by typsinization and centrifugation. The harvested cells were re-suspended in the number of 1×10⁶ cells/ml in culture medium containing 2% FBS. Hoechst33342 dye (5 μg/ml, Invitrogen, Carlsbad, U.S.A.) was added with or without reserpine (50 μM, Sigma, St. Louis, U.S.A.) as a blocking reagent. The cells were further incubated at 37° C. for 2 hours under frequently gentle vortex to ensure uniform staining, and then washed cells with PBS. PI staining (20 ng/ml) was used to determine the live/dead cells. In order to set up the fluorescence compensation, single staining and non-staining groups were prepared. The cell samples were filtered with nylon mesh (35 μm) to prevent cell aggregation. PI-positive dead cells were primary excluded to avoid false positive signals. Then, the Hoechst blue and red dot plotting was used to determine the side population. A gate of a side population was defined by the diminishing region of two samples groups, i.e. with or without reserpine. The percentages of the side population were further analyzed by Flowjo software (TreeStar, Ashland, U.S.A.). The results are shown in FIGS. 7A and 7B.

With reference to FIGS. 7A and 7B, they show characterization of side population (SP) in A549 cells treated with the extract of C. chinensis and the berberine solution, respectively. In FIGS. 7A and 7B, it can be seen that the side population is considerably diminished after the treatment of the extract of C. chinensis and the berberine solution. Referring to FIGS. 7A, 7B, and 5F to 5H, it can be understood that the extract of C. chinensis is able to inhibit cancer stem cells.

Although the present invention has been explained in relation to its preferred embodiment, it is to be understood that many other possible modifications and variations can be made without departing from the spirit and scope of the invention as hereinafter claimed.

What is claimed is:

1. A method for treating cancer or carcinoma metastasis, comprising:
administering an effective amount of a berberine compound to a subject in need thereof.

2. The method of claim 1, wherein the berberine compound is berberine chloride.

3. The method of claim 1, wherein the berberine compound is contained in an extract of a Coptis chinensis herbal material.

4. The method of claim 3, wherein the extract of the C. chinensis herbal material is a water extract of the C. chinensis herbal material or obtained by drying the water extract of the C. chinensis herbal material.

5. The method of claim 4, wherein the water extract of the C. chinensis herbal material is obtained by the following steps:
providing a herbal material of C. chinensis;
mixing water with the herbal material of C. chinensis to form a mixture, wherein the amount of water is 50-200 times the weight of the herbal material of C. chinensis; and
extracting the mixture under heating.

6. The method of claim 5, wherein the time of the extraction is in a range from 30 minutes to 2 hours.

7. The method of claim 5, wherein the mixture is heated until the volume of the mixture is changed into one-fourth to half of the original volume of the mixture.

8. The method of claim 1, wherein the cancer stem cells or the carcinoma cells are non-small-cell lung carcinoma (NSCLC) cells.

9. The method of claim 1, wherein administration of the berberine compound to the subject results in inhibition of cancer stem cells in the subject.
